(19) United States
(12) Patent Application Publication
(10) Pub. No.: US 2026/0268888 A1
(43) Pub. Date: Sep. 10, 2026

(54) SYSTEM

(71) Applicant: SoftBank Group Corp., Tokyo (JP)

(72) Inventor: Masahiro MIYAKE, Tokyo (JP)

(21) Appl. No.: 19/073,219

(22) Filed: Mar. 7, 2025

(51) Int. Cl. G10L 13/047; G16H 80/00

(52) U.S. Cl. CPC G10L 13/047; G16H 80/00

(57) ABSTRACT

The system according to the embodiment comprises a collection unit, an analysis unit, a generation unit, and a speech unit. The collection unit collects vital information. The analysis unit analyzes the information collected by the collection unit to understand emotions or thoughts. The generation unit generates words or sentences based on the analysis results obtained by the analysis unit. The speech unit articulates the words or sentences generated by the generation unit.

### BACKGROUND OF THE INVENTION

### Field of the Invention

The technology of this disclosure relates to a system.

### Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there was a problem that understanding emotions and thoughts based on vital information and generating and articulating appropriate words or sentences were not sufficiently performed.

### SUMMARY OF THE INVENTION

A system according to an embodiment comprises a collection unit, an analysis unit, a generation unit, and a speech unit. The collection unit collects vital information. The analysis unit analyzes the information collected by the collection unit to understand emotions or thoughts. The generation unit generates words or sentences based on the analysis results obtained by the analysis unit. The speech unit articulates the words or sentences generated by the generation unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset- type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor with a sign (hereinafter simply referred to as "processor") may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) with a sign is a memory where information is temporarily stored and used as a work memory by the processor.

In the following embodiments, a storage with a sign is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) with a sign is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, "A and/or B" means "at least one of A and B." In other words, "A and/or B" means it may be only A, only B, or a combination of A and B. Moreover, when expressing three or more items connected by "and/or," the same concept as "A and/or B" applies.

### First Embodiment

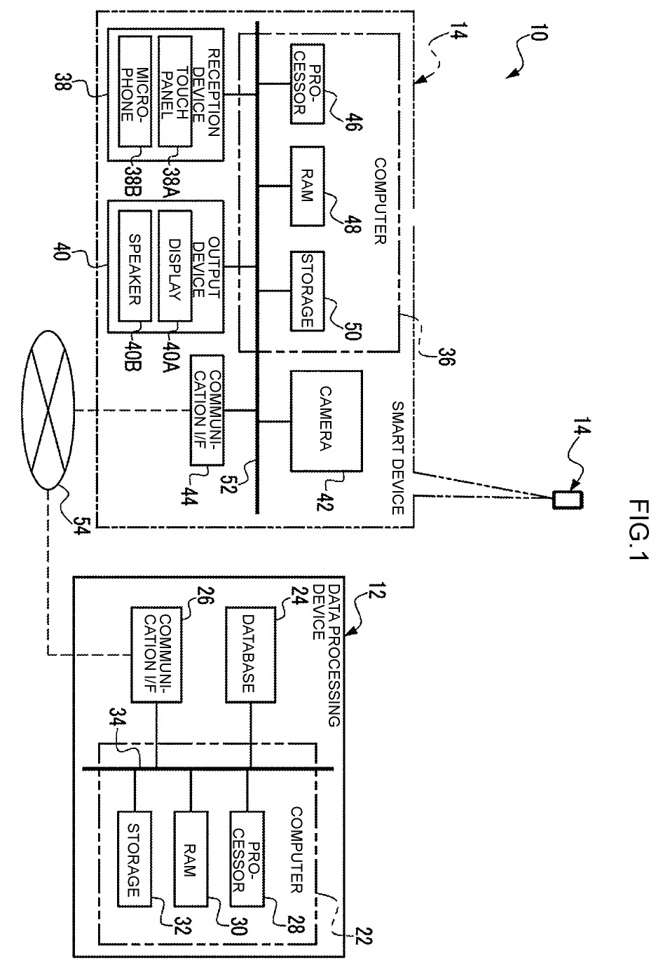

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

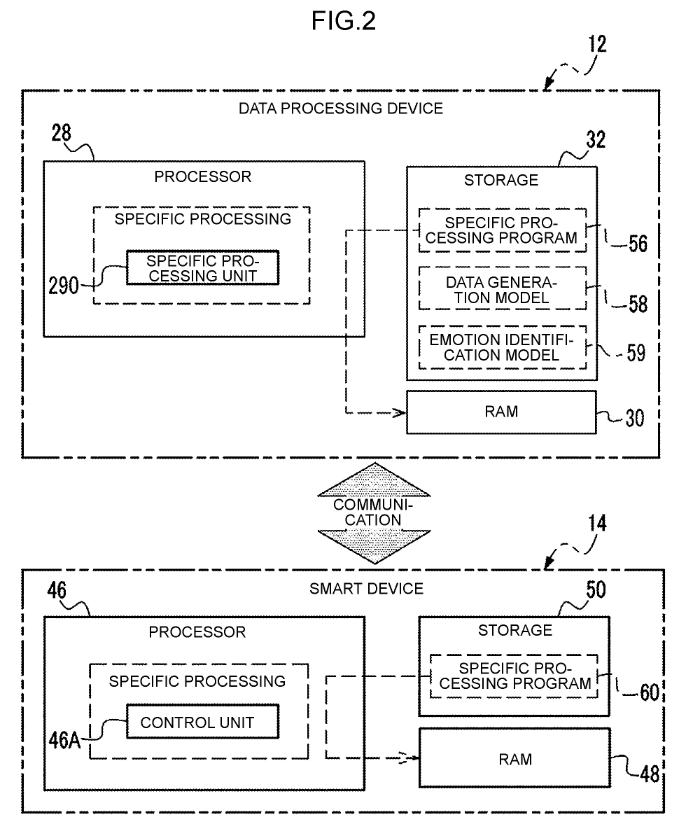

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide- Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a "program" related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59 and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1 of the Embodiment

The communication system according to the embodiment of the present invention collects vital information, understands the person's emotions and thoughts, generates words or sentences using generative AI, and articulates them using voice generative AI. This system analyzes the collected vital information and generates words or sentences using generative AI. Furthermore, by articulating them using voice generative AI, communication can be achieved. The intended users are expected to include ALS patients, intubated individuals who cannot speak, and people with different native languages, expanding communication possibilities. For example, an ALS patient wears a brain wave sensor to have their emotions and thoughts analyzed. The generative AI generates words like "I want to drink water," and the voice generative AI articulates those words. This allows the patient to convey their intentions to others. Additionally, when people with different native languages communicate, the generative AI generates appropriate words or sentences, and the voice generative AI articulates them, enabling communication beyond language barriers. This allows the communication system to convey the user's emotions and thoughts to others.

The communication system according to the embodiment comprises a collection unit, an analysis unit, a generation unit, and a speech unit. The collection unit collects vital information, which includes, for example, heart rate, brain waves, and body temperature, but is not limited to these examples. The collection unit collects vital information using, for example, brain wave sensors or heartbeat sensors. Brain wave sensors such as EEG sensors or fNIRS sensors are used. Heartbeat sensors such as photoplethysmographic sensors or electrical heartbeat sensors are used. The analysis unit uses generative AI to analyze the vital information collected by the collection unit and understand emotions and thoughts. Generative AI such as deep learning models or natural language processing models are used. The generation unit uses generative AI to generate words or sentences based on the analysis results obtained by the analysis unit. Generative AI such as text generation AI (e.g., LLM) or multimodal generation AI are used. The speech unit uses voice generative AI to articulate the words or sentences generated by the generation unit. Voice generative AI such as voice synthesis models or text-to-speech conversion technologies are used. This allows the communication system according to the embodiment to convey the user's emotions and thoughts to others.

The collection unit collects vital information, which includes, for example, heart rate, brain waves, and body temperature, but is not limited to these examples. The collection unit collects vital information using, for example, brain wave sensors or heartbeat sensors. Brain wave sensors such as EEG sensors or fNIRS sensors are used. EEG sensors measure brain electrical activity by being attached to the scalp, obtaining real-time brain wave data. fNIRS sensors measure changes in brain blood flow using near-infrared light, understanding brain activity states. Heartbeat sensors such as photoplethysmographic sensors or electrical heartbeat sensors are used. Photoplethysmographic sensors detect heart rate by measuring reflected light on the skin. Electrical heartbeat sensors detect heart rate by measuring the heart's electrical activity with electrodes attached to the skin. These sensors are incorporated into wearable devices or medical equipment, allowing continuous monitoring of the user's vital information. The collected vital information is transmitted to a central database using wireless communication technology and provided to the analysis unit in real-time. This allows the collection unit to accurately grasp the user's health and emotional state and provide the necessary data for the next analysis step. Furthermore, the collection unit can integrate data from multiple sensors, perform noise removal, and filter out abnormal values to improve data accuracy and reliability. This allows the collection unit to provide more accurate vital information and enhance the overall system performance.

The analysis unit uses generative AI to analyze the vital information collected by the collection unit and understand emotions and thoughts. Generative AI such as deep learning models or natural language processing models are used. Specifically, deep learning models classify the user's emotional state by inputting collected brain wave data or heartbeat data. For example, brain wave data is analyzed for alpha and beta wave patterns to identify relaxation or concentration states. Heartbeat data is analyzed for heart rate variability to assess stress levels or excitement states. Natural language processing models express the user's emotions and thoughts in text format based on these analysis results. For example, it outputs specific emotional states like "The user is currently relaxed." Furthermore, the analysis unit can utilize past data and user history information to analyze emotional changes and trends. This allows for understanding the user's long-term emotional patterns and providing more accurate analysis results. The analysis unit can also use anomaly detection algorithms to detect unusual emotional states or abnormal vital data and issue early warnings. This allows the analysis unit to not only perform real-time emotional analysis but also manage long-term emotions and detect anomalies, enhancing the system's reliability and safety.

The generation unit uses generative AI to generate words or sentences based on the analysis results obtained by the analysis unit. Generative AI such as text generation AI (e.g., LLM) or multimodal generation AI are used. Text generation AI generates natural words or sentences by inputting emotional states or thoughts provided by the analysis unit. For example, it generates specific sentences like "The user is currently relaxed and feeling calm." Multimodal generation AI can integrate not only text but also other modalities like images and sounds to generate richer expressions. For example, it generates images or audio messages corresponding to the user's emotional state, conveying emotions through visual and auditory means. The generation unit outputs the generated words or sentences in an appropriate format and provides them to the speech unit. Furthermore, the generation unit can collect user feedback and continuously improve the generative AI model. For example, users can evaluate generated sentences as "accurate" or "inaccurate," improving the generative AI's accuracy. Additionally, the generation unit can generate customized sentences according to specific situations or contexts. This allows the generation unit to accurately and naturally express the user's emotions and thoughts, providing information to convey to others.

The speech unit uses voice generative AI to articulate the words or sentences generated by the generation unit. Voice generative AI such as voice synthesis models or text-to-speech conversion technologies are used. Voice synthesis models generate natural voice by inputting text provided by the generation unit. For example, it generates voice with tones and intonations corresponding to the user's emotional state, effectively conveying emotions. Text-to-speech conversion technology adjusts pronunciation and accent when converting text to voice, generating easily understandable voice. The speech unit plays the generated voice through speakers or headsets, conveying the user's emotions and thoughts to others. Furthermore, the speech unit can collect user feedback and continuously improve the voice generative AI model. For example, users can evaluate generated voice as "easy to understand" or "difficult to understand," improving the voice generative AI's accuracy. Additionally, the speech unit can generate customized voice according to specific situations or contexts. This allows the speech unit to accurately and naturally express the user's emotions and thoughts in voice, providing information to convey to others.

The collection unit can include a brain wave sensor or a heartbeat sensor. Brain wave sensors include, for example, EEG sensors or fNIRS sensors. EEG sensors are sensors that electrically measure brain waves, and fNIRS sensors are sensors that measure brain blood flow using near-infrared light. Heartbeat sensors include, for example, photoplethysmographic sensors or electrical heartbeat sensors. Photoplethysmographic sensors are sensors that measure blood flow using light, and electrical heartbeat sensors are sensors that electrically measure heartbeats. This allows the collection unit to collect more accurate vital information using brain wave sensors or heartbeat sensors. Some or all of the aforementioned processing in the collection unit may be performed using AI or may be performed without using AI.

The analysis unit can analyze emotions or thoughts using generative AI. Generative AI includes, for example, deep learning models or natural language processing models. Deep learning models are models that learn from large amounts of data and have advanced analysis capabilities. Natural language processing models are models that analyze text data and understand its meaning. This allows the analysis unit to improve the accuracy of emotion and thought analysis by using generative AI. Some or all of the aforementioned processing in the analysis unit is performed using generative AI.

The generation unit can generate words or sentences using generative AI. Generative AI includes, for example, text generation AI (e.g., LLM) or multimodal generation AI. Text generation AI is a model that learns from large amounts of text data and has advanced natural language processing capabilities. Multimodal generation AI is a model that can handle multiple modalities, including text, images, and sounds. This allows the generation unit to improve the accuracy of word and sentence generation by using generative AI. Some or all of the aforementioned processing in the generation unit is performed using generative AI.

The speech unit can articulate words or sentences using voice generative AI. Voice generative AI includes, for example, voice synthesis models or text-to-speech conversion technologies. Voice synthesis models are models that convert text data into voice, and text-to-speech conversion technologies are technologies that convert text data into voice. This allows the speech unit to improve the accuracy of articulation by using voice generative AI. Some or all of the aforementioned processing in the speech unit is performed using voice generative AI.

The collection unit can collect brain waves using a brain wave sensor. Brain wave sensors include, for example, EEG sensors or fNIRS sensors. EEG sensors are sensors that electrically measure brain waves, and fNIRS sensors are sensors that measure brain blood flow using near-infrared light. This allows the collection unit to collect brain waves using brain wave sensors. Some or all of the aforementioned processing in the collection unit may be performed using AI or may be performed without using AI.

The collection unit can collect heartbeats using a heartbeat sensor. Heartbeat sensors include, for example, photoplethysmographic sensors or electrical heartbeat sensors. Photoplethysmographic sensors are sensors that measure blood flow using light, and electrical heartbeat sensors are sensors that electrically measure heartbeats. This allows the collection unit to collect heartbeats using heartbeat sensors. Some or all of the aforementioned processing in the collection unit may be performed using AI or may be performed without using AI.

The collection unit can analyze the user's past vital information and select an appropriate collection method. For example, the collection unit selects the most stable collection timing based on the user's past vital information. Additionally, the collection unit can concentrate collection at specific times based on the user's past vital information. Furthermore, the collection unit can analyze the user's past vital information and determine the optimal sensor placement. This allows the collection unit to select the optimal collection method by analyzing past vital information. Some or all of the aforementioned processing in the collection unit may be performed using AI or may be performed without using AI.

The collection unit can filter based on the user's current health status or activity level during vital information collection. For example, the collection unit prioritizes collecting vital information related to exercise when the user is exercising. Additionally, the collection unit can collect vital information related to relaxation when the user is resting. Furthermore, the collection unit can collect detailed vital information related to health status when the user is ill. This allows the collection unit to collect more relevant vital information by filtering based on health status or activity level. Some or all of the aforementioned processing in the collection unit may be performed using AI or may be performed without using AI.

The collection unit can prioritize collecting relevant information by considering the user's geographical location during vital information collection. For example, the collection unit prioritizes collecting oxygen saturation and respiration rate when the user is at high altitude. Additionally, the collection unit can prioritize collecting stress-related vital information when the user is in urban areas. Furthermore, the collection unit can prioritize collecting relaxation-related vital information when the user is in natural environments. This allows the collection unit to collect relevant vital information by considering geographical location. Some or all of the aforementioned processing in the collection unit may be performed using AI or may be performed without using AI.

The collection unit can analyze the user's social media activity and collect relevant information during vital information collection. For example, the collection unit prioritizes collecting stress-related vital information when the user feels stressed on social media. Additionally, the collection unit can prioritize collecting relaxation-related vital information when the user is relaxed on social media. Furthermore, the collection unit can prioritize collecting excitement-related vital information when the user is excited on social media. This allows the collection unit to collect relevant vital information by analyzing social media activity. Some or all of the aforementioned processing in the collection unit may be performed using AI or may be performed without using AI.

The analysis unit can adjust the level of detail of analysis based on the importance of vital information during analysis. For example, the analysis unit performs detailed analysis on highly important vital information. Additionally, the analysis unit can perform simplified analysis on less important vital information. Furthermore, the analysis unit can perform analysis with moderate detail on moderately important vital information. This allows the analysis unit to perform efficient analysis by adjusting the level of detail based on importance. Some or all of the aforementioned processing in the analysis unit may be performed using AI or may be performed without using AI.

The analysis unit can apply different analysis algorithms according to the category of vital information during analysis. For example, the analysis unit applies heart rate analysis algorithms to vital information related to heart rate. Additionally, the analysis unit can apply brain wave analysis algorithms to vital information related to brain waves. Furthermore, the analysis unit can apply respiration analysis algorithms to vital information related to respiration rate. This allows the analysis unit to improve analysis accuracy by applying analysis algorithms according to categories. Some or all of the aforementioned processing in the analysis unit may be performed using AI or may be performed without using AI.

The analysis unit can determine the priority of analysis based on the collection timing of vital information during analysis. For example, the analysis unit prioritizes analyzing the latest vital information. Additionally, the analysis unit can analyze current vital information while referring to past vital information. Furthermore, the analysis unit can prioritize analyzing vital information collected at specific times. This allows the analysis unit to prioritize analyzing the latest information by determining the priority based on collection timing. Some or all of the aforementioned processing in the analysis unit may be performed using AI or may be performed without using AI.

The analysis unit can adjust the order of analysis based on the relevance of vital information during analysis. For example, the analysis unit prioritizes analyzing highly relevant vital information. Additionally, the analysis unit can postpone analyzing less relevant vital information. Furthermore, the analysis unit can analyze moderately relevant vital information in an appropriate order. This allows the analysis unit to perform efficient analysis by adjusting the order based on relevance. Some or all of the aforementioned processing in the analysis unit may be performed using AI or may be performed without using AI.

The generation unit can adjust the level of detail of generation based on the intensity of emotions during the generation of words or sentences. For example, the generation unit generates detailed words or sentences when the intensity of emotions is high. Additionally, the generation unit can generate concise words or sentences when the intensity of emotions is low. Furthermore, the generation unit can generate words or sentences with moderate detail when the intensity of emotions is moderate. This allows the generation unit to generate appropriately detailed words or sentences by adjusting the level of detail based on the intensity of emotions. Some or all of the aforementioned processing in the generation unit may be performed using generative AI or may be performed without using generative AI.

The generation unit can apply different generation algorithms according to the type of emotions during the generation of words or sentences. For example, the generation unit applies positive expression generation algorithms to joyful emotions. Additionally, the generation unit can apply soothing expression generation algorithms to sad emotions. Furthermore, the generation unit can apply calm expression generation algorithms to angry emotions. This allows the generation unit to generate appropriately expressed words or sentences by applying generation algorithms according to the type of emotions. Some or all of the aforementioned processing in the generation unit may be performed using generative AI or may be performed without using generative AI.

The generation unit can determine the priority of generation based on the occurrence timing of emotions during the generation of words or sentences. For example, the generation unit prioritizes generating words or sentences based on the latest emotions. Additionally, the generation unit can generate words or sentences based on current emotions while referring to past emotions. Furthermore, the generation unit can generate words or sentences based on emotions that occurred at specific times. This allows the generation unit to prioritize generating words or sentences based on the latest emotions by determining the priority based on occurrence timing. Some or all of the aforementioned processing in the generation unit may be performed using generative AI or may be performed without using generative AI.

The generation unit can adjust the order of generation based on the relevance of emotions during the generation of words or sentences. For example, the generation unit prioritizes generating words or sentences based on highly relevant emotions. Additionally, the generation unit can postpone generating words or sentences based on less relevant emotions. Furthermore, the generation unit can generate words or sentences based on moderately relevant emotions in an appropriate order. This allows the generation unit to perform efficient generation by adjusting the order based on relevance. Some or all of the aforementioned processing in the generation unit may be performed using generative AI or may be performed without using generative AI.

The speech unit can adjust the level of detail of speech based on the importance of generated words or sentences during speech. For example, the speech unit articulates in detail for highly important words or sentences. Additionally, the speech unit can articulate concisely for less important words or sentences. Furthermore, the speech unit can articulate with moderate detail for moderately important words or sentences. This allows the speech unit to perform efficient speech by adjusting the level of detail based on importance. Some or all of the aforementioned processing in the speech unit may be performed using AI or may be performed without using AI.

The speech unit can apply different speech algorithms according to the category of generated words or sentences during speech. For example, the speech unit applies questioning speech algorithms to words or sentences related to questions. Additionally, the speech unit can apply clear and directive speech algorithms to words or sentences related to instructions. Furthermore, the speech unit can apply speech algorithms that convey gratitude to words or sentences related to appreciation. This allows the speech unit to perform appropriate speech by applying speech algorithms according to categories. Some or all of the aforementioned processing in the speech unit may be performed using AI or may be performed without using AI.

The speech unit can determine the priority of speech based on the occurrence timing of generated words or sentences during speech. For example, the speech unit prioritizes articulating the latest words or sentences. Additionally, the speech unit can articulate current words or sentences while referring to past words or sentences. Furthermore, the speech unit can prioritize articulating words or sentences generated at specific times. This allows the speech unit to prioritize articulating the latest words or sentences by determining the priority based on occurrence timing. Some or all of the aforementioned processing in the speech unit may be performed using AI or may be performed without using AI.

The speech unit can adjust the order of speech based on the relevance of generated words or sentences during speech. For example, the speech unit prioritizes articulating highly relevant words or sentences. Additionally, the speech unit can postpone articulating less relevant words or sentences. Furthermore, the speech unit can articulate moderately relevant words or sentences in an appropriate order. This allows the speech unit to perform efficient speech by adjusting the order based on relevance. Some or all of the aforementioned processing in the speech unit may be performed using AI or may be performed without using AI.

The system according to the embodiment is not limited to the examples described above, and various modifications are possible, such as the following.

The communication system can further include a history analysis unit that analyzes the user's past communication history and generates appropriate words or sentences. For example, it learns phrases and expressions frequently used by the user in the past and generates natural conversations based on them. Additionally, the history analysis unit can consider the user's past emotional states and provide appropriate expressions at the right timing. Furthermore, the history analysis unit can analyze the user's past communication patterns and prepare anticipated questions and responses in advance. This allows the communication system to utilize the user's past communication history for more natural and effective communication.

The generation unit can further generate words or sentences considering the user's preferences and interests. For example, when the user is interested in a specific topic, it generates words or sentences related to that topic. Additionally, the generation unit can learn the user's preferred expressions and styles based on past statements and actions and generate words or sentences reflecting them. Furthermore, the generation unit can consider the user's current situation and context to generate appropriate words or sentences. This allows the generation unit to perform communication reflecting the user's preferences and interests.

The speech unit can further learn the user's voice characteristics and generate personalized voice. For example, it learns the user's voice pitch, tone, and rhythm and generates natural voice based on them. Additionally, the speech unit can generate voice reflecting the user's voice characteristics, providing voice close to the user's own voice. Furthermore, the speech unit can generate voice corresponding to different emotional states based on the user's voice characteristics. This allows the speech unit to perform personalized voice generation reflecting the user's voice characteristics.

The generation unit can further generate words or sentences considering the user's cultural background. For example, when the user belongs to a specific cultural sphere, it generates expressions and words suitable for that culture. Additionally, the generation unit can consider the user's religion and customs to generate appropriate words or sentences. Furthermore, the generation unit can learn region-specific phrases and slang and generate words or sentences reflecting them. This allows the generation unit to perform communication considering cultural background.

The speech unit can further generate voice considering the user's auditory characteristics. For example, when the user has difficulty hearing high frequencies, it generates voice emphasizing low frequencies. Additionally, the speech unit can generate voice emphasizing frequency bands the user can easily hear. Furthermore, the speech unit can adjust the speed and rhythm of voice according to the user's auditory characteristics. This allows the speech unit to perform voice generation considering auditory characteristics.

Below is a brief explanation of the process flow in Example 1 of the Embodiment.

Step 1: The collection unit collects vital information. Vital information includes heart rate, brain waves, and body temperature. The collection unit collects vital information using brain wave sensors or heartbeat sensors. EEG sensors or fNIRS sensors are used as brain wave sensors, and photoplethysmographic sensors or electrical heartbeat sensors are used as heartbeat sensors.

Step 2: The analysis unit uses generative AI to analyze the vital information collected by the collection unit and understand emotions and thoughts. Deep learning models or natural language processing models are used as generative AI.

Step 3: The generation unit uses generative AI to generate words or sentences based on the analysis results obtained by the analysis unit. Text generation AI (e.g., LLM) or multimodal generation AI are used as generative AI.

Step 4: The speech unit uses voice generative AI to articulate the words or sentences generated by the generation unit. Voice synthesis models or text-to-speech conversion technologies are used as voice generative AI.

### Example 2 of the Embodiment

The communication system according to the embodiment of the present invention collects vital information, understands the person's emotions and thoughts, generates words or sentences using generative AI, and articulates them using voice generative AI. This system analyzes the collected vital information and generates words or sentences using generative AI. Furthermore, by articulating them using voice generative AI, communication can be achieved. The intended users are expected to include ALS patients, intubated individuals who cannot speak, and people with different native languages, expanding communication possibilities. For example, an ALS patient wears a brain wave sensor to have their emotions and thoughts analyzed. The generative AI generates words like "I want to drink water," and the voice generative AI articulates those words. This allows the patient to convey their intentions to others. Additionally, when people with different native languages communicate, the generative AI generates appropriate words or sentences, and the voice generative AI articulates them, enabling communication beyond language barriers. This allows the communication system to convey the user's emotions and thoughts to others.

The communication system according to the embodiment comprises a collection unit, an analysis unit, a generation unit, and a speech unit. The collection unit collects vital information, which includes, for example, heart rate, brain waves, and body temperature, but is not limited to these examples. The collection unit collects vital information using, for example, brain wave sensors or heartbeat sensors. Brain wave sensors such as EEG sensors or fNIRS sensors are used. Heartbeat sensors such as photoplethysmographic sensors or electrical heartbeat sensors are used. The analysis unit uses generative AI to analyze the vital information collected by the collection unit and understand emotions and thoughts. Generative AI such as deep learning models or natural language processing models are used. The generation unit uses generative AI to generate words or sentences based on the analysis results obtained by the analysis unit. Generative AI such as text generation AI (e.g., LLM) or multimodal generation AI are used. The speech unit uses voice generative AI to articulate the words or sentences generated by the generation unit. Voice generative AI such as voice synthesis models or text-to-speech conversion technologies are used. This allows the communication system according to the embodiment to convey the user's emotions and thoughts to others.

The collection unit collects vital information, which includes, for example, heart rate, brain waves, and body temperature, but is not limited to these examples. The collection unit collects vital information using, for example, brain wave sensors or heartbeat sensors. Brain wave sensors such as EEG sensors or fNIRS sensors are used. EEG sensors measure brain electrical activity by being attached to the scalp, obtaining real-time brain wave data. fNIRS sensors measure changes in brain blood flow using near-infrared light, understanding brain activity states. Heartbeat sensors such as photoplethysmographic sensors or electrical heartbeat sensors are used. Photoplethysmographic sensors detect heart rate by measuring reflected light on the skin. Electrical heartbeat sensors detect heart rate by measuring the heart's electrical activity with electrodes attached to the skin. These sensors are incorporated into wearable devices or medical equipment, allowing continuous monitoring of the user's vital information. The collected vital information is transmitted to a central database using wireless communication technology and provided to the analysis unit in real-time. This allows the collection unit to accurately grasp the user's health and emotional state and provide the necessary data for the next analysis step. Furthermore, the collection unit can integrate data from multiple sensors, perform noise removal, and filter out abnormal values to improve data accuracy and reliability. This allows the collection unit to provide more accurate vital information and enhance the overall system performance.

The analysis unit uses generative AI to analyze the vital information collected by the collection unit and understand emotions and thoughts. Generative AI such as deep learning models or natural language processing models are used. Specifically, deep learning models classify the user's emotional state by inputting collected brain wave data or heartbeat data. For example, brain wave data is analyzed for alpha and beta wave patterns to identify relaxation or concentration states. Heartbeat data is analyzed for heart rate variability to assess stress levels or excitement states. Natural language processing models express the user's emotions and thoughts in text format based on these analysis results. For example, it outputs specific emotional states like "The user is currently relaxed." Furthermore, the analysis unit can utilize past data and user history information to analyze emotional changes and trends. This allows for understanding the user's long-term emotional patterns and providing more accurate analysis results. The analysis unit can also use anomaly detection algorithms to detect unusual emotional states or abnormal vital data and issue early warnings. This allows the analysis unit to not only perform real-time emotional analysis but also manage long-term emotions and detect anomalies, enhancing the system's reliability and safety.

The generation unit uses generative AI to generate words or sentences based on the analysis results obtained by the analysis unit. Generative AI such as text generation AI (e.g., LLM) or multimodal generation AI are used. Text generation AI generates natural words or sentences by inputting emotional states or thoughts provided by the analysis unit. For example, it generates specific sentences like "The user is currently relaxed and feeling calm." Multimodal generation AI can integrate not only text but also other modalities like images and sounds to generate richer expressions. For example, it generates images or audio messages corresponding to the user's emotional state, conveying emotions through visual and auditory means. The generation unit outputs the generated words or sentences in an appropriate format and provides them to the speech unit. Furthermore, the generation unit can collect user feedback and continuously improve the generative AI model. For example, users can evaluate generated sentences as "accurate" or "inaccurate," improving the generative AI's accuracy. Additionally, the generation unit can generate customized sentences according to specific situations or contexts. This allows the generation unit to accurately and naturally express the user's emotions and thoughts, providing information to convey to others.

The speech unit uses voice generative AI to articulate the words or sentences generated by the generation unit. Voice generative AI such as voice synthesis models or text- to-speech conversion technologies are used. Voice synthesis models generate natural voice by inputting text provided by the generation unit. For example, it generates voice with tones and intonations corresponding to the user's emotional state, effectively conveying emotions. Text-to-speech conversion technology adjusts pronunciation and accent when converting text to voice, generating easily understandable voice. The speech unit plays the generated voice through speakers or headsets, conveying the user's emotions and thoughts to others. Furthermore, the speech unit can collect user feedback and continuously improve the voice generative AI model. For example, users can evaluate generated voice as "easy to understand" or "difficult to understand," improving the voice generative AI's accuracy. Additionally, the speech unit can generate customized voice according to specific situations or contexts. This allows the speech unit to accurately and naturally express the user's emotions and thoughts in voice, providing information to convey to others.

The collection unit can include a brain wave sensor or a heartbeat sensor. Brain wave sensors include, for example, EEG sensors or fNIRS sensors. EEG sensors are sensors that electrically measure brain waves, and fNIRS sensors are sensors that measure brain blood flow using near-infrared light. Heartbeat sensors include, for example, photoplethysmographic sensors or electrical heartbeat sensors. Photoplethysmographic sensors are sensors that measure blood flow using light, and electrical heartbeat sensors are sensors that electrically measure heartbeats. This allows the collection unit to collect more accurate vital information using brain wave sensors or heartbeat sensors. Some or all of the aforementioned processing in the collection unit may be performed using AI or may be performed without using AI.

The analysis unit can analyze emotions or thoughts using generative AI. Generative AI includes, for example, deep learning models or natural language processing models. Deep learning models are models that learn from large amounts of data and have advanced analysis capabilities. Natural language processing models are models that analyze text data and understand its meaning. This allows the analysis unit to improve the accuracy of emotion and thought analysis by using generative AI. Some or all of the aforementioned processing in the analysis unit is performed using generative AI.

The generation unit can generate words or sentences using generative AI. Generative AI includes, for example, text generation AI (e.g., LLM) or multimodal generation AI. Text generation AI is a model that learns from large amounts of text data and has advanced natural language processing capabilities. Multimodal generation AI is a model that can handle multiple modalities, including text, images, and sounds. This allows the generation unit to improve the accuracy of word and sentence generation by using generative AI. Some or all of the aforementioned processing in the generation unit is performed using generative AI.

The speech unit can articulate words or sentences using voice generative AI. Voice generative AI includes, for example, voice synthesis models or text-to-speech conversion technologies. Voice synthesis models are models that convert text data into voice, and text-to-speech conversion technologies are technologies that convert text data into voice. This allows the speech unit to improve the accuracy of articulation by using voice generative AI. Some or all of the aforementioned processing in the speech unit is performed using voice generative AI.

The collection unit can collect brain waves using a brain wave sensor. Brain wave sensors include, for example, EEG sensors or fNIRS sensors. EEG sensors are sensors that electrically measure brain waves, and fNIRS sensors are sensors that measure brain blood flow using near-infrared light. This allows the collection unit to collect brain waves using brain wave sensors. Some or all of the aforementioned processing in the collection unit may be performed using AI or may be performed without using AI.

The collection unit can collect heartbeats using a heartbeat sensor. Heartbeat sensors include, for example, photoplethysmographic sensors or electrical heartbeat sensors. Photoplethysmographic sensors are sensors that measure blood flow using light, and electrical heartbeat sensors are sensors that electrically measure heartbeats. This allows the collection unit to collect heartbeats using heartbeat sensors. Some or all of the aforementioned processing in the collection unit may be performed using AI or may be performed without using AI.

The collection unit can estimate the user's emotions and adjust the timing of vital information collection based on the estimated emotions. For example, the collection unit sets a low collection frequency and collects only the minimum necessary data when the user is relaxed. Additionally, the collection unit can set a high collection frequency and collect detailed data when the user is stressed. Furthermore, the collection unit can approach real-time collection timing and collect immediate data when the user is excited. This allows the collection unit to collect more appropriate vital information by adjusting the collection timing based on the user's emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the collection unit may be performed using AI or may be performed without using AI.

The collection unit can analyze the user's past vital information and select an appropriate collection method. For example, the collection unit selects the most stable collection timing based on the user's past vital information. Additionally, the collection unit can concentrate collection at specific times based on the user's past vital information. Furthermore, the collection unit can analyze the user's past vital information and determine the optimal sensor placement. This allows the collection unit to select the optimal collection method by analyzing past vital information. Some or all of the aforementioned processing in the collection unit may be performed using AI or may be performed without using AI.

The collection unit can filter based on the user's current health status or activity level during vital information collection. For example, the collection unit prioritizes collecting vital information related to exercise when the user is exercising. Additionally, the collection unit can collect vital information related to relaxation when the user is resting. Furthermore, the collection unit can collect detailed vital information related to health status when the user is ill. This allows the collection unit to collect more relevant vital information by filtering based on health status or activity level. Some or all of the aforementioned processing in the collection unit may be performed using AI or may be performed without using AI.

The collection unit can estimate the user's emotions and determine the priority of vital information to be collected based on the estimated emotions. For example, the collection unit prioritizes collecting stress-related vital information such as heart rate and blood pressure when the user is tense. Additionally, the collection unit can prioritize collecting relaxation-related vital information such as brain waves and respiration rate when the user is relaxed. Furthermore, the collection unit can prioritize collecting excitement-related vital information such as brain waves and heart rate when the user is excited. This allows the collection unit to prioritize collecting important vital information by determining the priority based on emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the collection unit may be performed using AI or may be performed without using AI.

The collection unit can prioritize collecting relevant information by considering the user's geographical location during vital information collection. For example, the collection unit prioritizes collecting oxygen saturation and respiration rate when the user is at high altitude. Additionally, the collection unit can prioritize collecting stress-related vital information when the user is in urban areas. Furthermore, the collection unit can prioritize collecting relaxation-related vital information when the user is in natural environments. This allows the collection unit to collect relevant vital information by considering geographical location. Some or all of the aforementioned processing in the collection unit may be performed using AI or may be performed without using AI.

The collection unit can analyze the user's social media activity and collect relevant information during vital information collection. For example, the collection unit prioritizes collecting stress-related vital information when the user feels stressed on social media. Additionally, the collection unit can prioritize collecting relaxation-related vital information when the user is relaxed on social media. Furthermore, the collection unit can prioritize collecting excitement-related vital information when the user is excited on social media. This allows the collection unit to collect relevant vital information by analyzing social media activity. Some or all of the aforementioned processing in the collection unit may be performed using AI or may be performed without using AI.

The analysis unit can estimate the user's emotions and adjust the expression method of analysis based on the estimated emotions. For example, the analysis unit provides simple and highly visible analysis results when the user is tense. Additionally, the analysis unit can provide detailed analysis results when the user is relaxed. Furthermore, the analysis unit can provide visually stimulating analysis results when the user is excited. This allows the analysis unit to provide easily understandable analysis results by adjusting the expression method based on emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the analysis unit may be performed using AI or may be performed without using AI.

The analysis unit can adjust the level of detail of analysis based on the importance of vital information during analysis. For example, the analysis unit performs detailed analysis on highly important vital information. Additionally, the analysis unit can perform simplified analysis on less important vital information. Furthermore, the analysis unit can perform analysis with moderate detail on moderately important vital information. This allows the analysis unit to perform efficient analysis by adjusting the level of detail based on importance. Some or all of the aforementioned processing in the analysis unit may be performed using AI or may be performed without using AI.

The analysis unit can apply different analysis algorithms according to the category of vital information during analysis. For example, the analysis unit applies heart rate analysis algorithms to vital information related to heart rate. Additionally, the analysis unit can apply brain wave analysis algorithms to vital information related to brain waves. Furthermore, the analysis unit can apply respiration analysis algorithms to vital information related to respiration rate. This allows the analysis unit to improve analysis accuracy by applying analysis algorithms according to categories. Some or all of the aforementioned processing in the analysis unit may be performed using AI or may be performed without using AI.

The analysis unit can estimate the user's emotions and adjust the length of analysis based on the estimated emotions. For example, the analysis unit provides short and concise analysis results when the user is in a hurry. Additionally, the analysis unit can provide detailed analysis results when the user is relaxed. Furthermore, the analysis unit can provide visually stimulating analysis results when the user is excited. This allows the analysis unit to provide appropriately lengthy analysis results by adjusting the length based on emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the analysis unit may be performed using AI or may be performed without using AI.

The analysis unit can determine the priority of analysis based on the collection timing of vital information during analysis. For example, the analysis unit prioritizes analyzing the latest vital information. Additionally, the analysis unit can analyze current vital information while referring to past vital information. Furthermore, the analysis unit can prioritize analyzing vital information collected at specific times. This allows the analysis unit to prioritize analyzing the latest information by determining the priority based on collection timing. Some or all of the aforementioned processing in the analysis unit may be performed using AI or may be performed without using AI.

The analysis unit can adjust the order of analysis based on the relevance of vital information during analysis. For example, the analysis unit prioritizes analyzing highly relevant vital information. Additionally, the analysis unit can postpone analyzing less relevant vital information. Furthermore, the analysis unit can analyze moderately relevant vital information in an appropriate order. This allows the analysis unit to perform efficient analysis by adjusting the order based on relevance. Some or all of the aforementioned processing in the analysis unit may be performed using AI or may be performed without using AI.

The generation unit can estimate the user's emotions and adjust the expression method of words or sentences to be generated based on the estimated emotions. For example, the generation unit generates words or sentences with calm expressions when the user is relaxed. Additionally, the generation unit can generate concise and clear expressions when the user is tense. Furthermore, the generation unit can generate expressions that emphasize emotions when the user is excited. This allows the generation unit to generate appropriately expressed words or sentences by adjusting the expression method based on emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the generation unit is performed using generative AI.

The generation unit can adjust the level of detail of generation based on the intensity of emotions during the generation of words or sentences. For example, the generation unit generates detailed words or sentences when the intensity of emotions is high. Additionally, the generation unit can generate concise words or sentences when the intensity of emotions is low. Furthermore, the generation unit can generate words or sentences with moderate detail when the intensity of emotions is moderate. This allows the generation unit to generate appropriately detailed words or sentences by adjusting the level of detail based on the intensity of emotions. Some or all of the aforementioned processing in the generation unit may be performed using generative AI or may be performed without using generative AI.

The generation unit can apply different generation algorithms according to the type of emotions during the generation of words or sentences. For example, the generation unit applies positive expression generation algorithms to joyful emotions. Additionally, the generation unit can apply soothing expression generation algorithms to sad emotions. Furthermore, the generation unit can apply calm expression generation algorithms to angry emotions. This allows the generation unit to generate appropriately expressed words or sentences by applying generation algorithms according to the type of emotions. Some or all of the aforementioned processing in the generation unit may be performed using generative AI or may be performed without using generative AI.

The generation unit can estimate the user's emotions and adjust the length of words or sentences to be generated based on the estimated emotions. For example, the generation unit generates short and concise words or sentences when the user is in a hurry. Additionally, the generation unit can generate longer words or sentences with detailed explanations when the user is relaxed. Furthermore, the generation unit can generate words or sentences with emphasized emotions when the user is excited. This allows the generation unit to generate appropriately lengthy words or sentences by adjusting the length based on emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the generation unit is performed using generative AI.

The generation unit can determine the priority of generation based on the occurrence timing of emotions during the generation of words or sentences. For example, the generation unit prioritizes generating words or sentences based on the latest emotions. Additionally, the generation unit can generate words or sentences based on current emotions while referring to past emotions. Furthermore, the generation unit can generate words or sentences based on emotions that occurred at specific times. This allows the generation unit to prioritize generating words or sentences based on the latest emotions by determining the priority based on occurrence timing. Some or all of the aforementioned processing in the generation unit may be performed using generative AI or may be performed without using generative AI.

The generation unit can adjust the order of generation based on the relevance of emotions during the generation of words or sentences. For example, the generation unit prioritizes generating words or sentences based on highly relevant emotions. Additionally, the generation unit can postpone generating words or sentences based on less relevant emotions. Furthermore, the generation unit can generate words or sentences based on moderately relevant emotions in an appropriate order. This allows the generation unit to perform efficient generation by adjusting the order based on relevance. Some or all of the aforementioned processing in the generation unit may be performed using generative AI or may be performed without using generative AI.

The speech unit can estimate the user's emotions and adjust the expression method of speech based on the estimated emotions. For example, the speech unit articulates in a calm voice when the user is relaxed. Additionally, the speech unit can articulate in a clear and composed voice when the user is tense. Furthermore, the speech unit can articulate in a voice that emphasizes emotions when the user is excited. This allows the speech unit to perform appropriately expressed speech by adjusting the expression method based on emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the speech unit may be performed using AI or may be performed without using AI.

The speech unit can adjust the level of detail of speech based on the importance of generated words or sentences during speech. For example, the speech unit articulates in detail for highly important words or sentences. Additionally, the speech unit can articulate concisely for less important words or sentences. Furthermore, the speech unit can articulate with moderate detail for moderately important words or sentences. This allows the speech unit to perform efficient speech by adjusting the level of detail based on importance. Some or all of the aforementioned processing in the speech unit may be performed using AI or may be performed without using AI.

The speech unit can apply different speech algorithms according to the category of generated words or sentences during speech. For example, the speech unit applies questioning speech algorithms to words or sentences related to questions. Additionally, the speech unit can apply clear and directive speech algorithms to words or sentences related to instructions. Furthermore, the speech unit can apply speech algorithms that convey gratitude to words or sentences related to appreciation. This allows the speech unit to perform appropriate speech by applying speech algorithms according to categories. Some or all of the aforementioned processing in the speech unit may be performed using AI or may be performed without using AI.

The speech unit can estimate the user's emotions and adjust the length of speech based on the estimated emotions. For example, the speech unit performs short and concise speech when the user is in a hurry. Additionally, the speech unit can perform longer speech with detailed explanations when the user is relaxed. Furthermore, the speech unit can perform speech with emphasized emotions when the user is excited. This allows the speech unit to perform appropriately lengthy speech by adjusting the length based on emotions. Emotion estimation is realized using, for example, an emotion engine or generative AI with emotion estimation functions. Generative AI includes text generation AI (e.g., LLM) or multimodal generation AI, but is not limited to these examples. Some or all of the aforementioned processing in the speech unit may be performed using AI or may be performed without using AI.

The speech unit can determine the priority of speech based on the occurrence timing of generated words or sentences during speech. For example, the speech unit prioritizes articulating the latest words or sentences. Additionally, the speech unit can articulate current words or sentences while referring to past words or sentences. Furthermore, the speech unit can prioritize articulating words or sentences generated at specific times. This allows the speech unit to prioritize articulating the latest words or sentences by determining the priority based on occurrence timing. Some or all of the aforementioned processing in the speech unit may be performed using AI or may be performed without using AI.

The speech unit can adjust the order of speech based on the relevance of generated words or sentences during speech. For example, the speech unit prioritizes articulating highly relevant words or sentences. Additionally, the speech unit can postpone articulating less relevant words or sentences. Furthermore, the speech unit can articulate moderately relevant words or sentences in an appropriate order. This allows the speech unit to perform efficient speech by adjusting the order based on relevance. Some or all of the aforementioned processing in the speech unit may be performed using AI or may be performed without using AI.

The system according to the embodiment is not limited to the examples described above, and various modifications are possible, such as the following.

The communication system can further include a history analysis unit that analyzes the user's past communication history and generates appropriate words or sentences. For example, it learns phrases and expressions frequently used by the user in the past and generates natural conversations based on them. Additionally, the history analysis unit can consider the user's past emotional states and provide appropriate expressions at the right timing. Furthermore, the history analysis unit can analyze the user's past communication patterns and prepare anticipated questions and responses in advance. This allows the communication system to utilize the user's past communication history for more natural and effective communication.

The collection unit can further collect the user's environmental sounds and analyze them in the analysis unit. For example, when the user is in a noisy environment, the collection unit collects the environmental sounds and performs noise filtering in the analysis unit. Additionally, the collection unit can collect environmental sounds when the user is in a quiet environment and estimate relaxation states in the analysis unit. Furthermore, the collection unit can collect specific sounds (e.g., music or natural sounds) the user is listening to and estimate the user's emotional state in the analysis unit. This allows the collection unit to consider environmental sounds for more accurate vital information analysis.

The analysis unit can further include a facial analysis unit that analyzes the user's facial expressions. For example, it captures the user's face using a camera and analyzes the expressions in the facial analysis unit. Additionally, the facial analysis unit can detect subtle facial changes and estimate emotional states. Furthermore, the facial analysis unit can analyze the user's eye movements and mouth movements to estimate more detailed emotional states. This allows the analysis unit to perform facial analysis for more accurate understanding of the user's emotional state.

The generation unit can further generate words or sentences considering the user's preferences and interests. For example, when the user is interested in a specific topic, it generates words or sentences related to that topic. Additionally, the generation unit can learn the user's preferred expressions and styles based on past statements and actions and generate words or sentences reflecting them. Furthermore, the generation unit can consider the user's current situation and context to generate appropriate words or sentences. This allows the generation unit to perform communication reflecting the user's preferences and interests.

The speech unit can further learn the user's voice characteristics and generate personalized voice. For example, it learns the user's voice pitch, tone, and rhythm and generates natural voice based on them. Additionally, the speech unit can generate voice reflecting the user's voice characteristics, providing voice close to the user's own voice. Furthermore, the speech unit can generate voice corresponding to different emotional states based on the user's voice characteristics. This allows the speech unit to perform personalized voice generation reflecting the user's voice characteristics.

The collection unit can further detect the user's body movements and collect vital information based on those movements. For example, when the user is exercising, it detects the movements and collects vital information related to exercise. Additionally, the collection unit can detect the user's movements when relaxing and collect vital information related to relaxation states. Furthermore, the collection unit can detect specific movements (e.g., raising hands, walking) the user is performing and collect appropriate vital information. This allows the collection unit to consider body movements for more accurate vital information collection.

The analysis unit can further analyze the user's voice and estimate emotional states from the voice. For example, it analyzes the user's voice tone, rhythm, and speed to estimate emotional states. Additionally, the analysis unit can analyze the user's voice intensity and intonation to estimate emotional states. Furthermore, the analysis unit can analyze changes in the user's voice in real-time and estimate emotional states immediately. This allows the analysis unit to perform voice analysis for more accurate understanding of the user's emotional state.

The generation unit can further generate words or sentences considering the user's cultural background. For example, when the user belongs to a specific cultural sphere, it generates expressions and words suitable for that culture. Additionally, the generation unit can consider the user's religion and customs to generate appropriate words or sentences. Furthermore, the generation unit can learn region-specific phrases and slang and generate words or sentences reflecting them. This allows the generation unit to perform communication considering cultural background.

The speech unit can further generate voice considering the user's auditory characteristics. For example, when the user has difficulty hearing high frequencies, it generates voice emphasizing low frequencies. Additionally, the speech unit can generate voice emphasizing frequency bands the user can easily hear. Furthermore, the speech unit can adjust the speed and rhythm of voice according to the user's auditory characteristics. This allows the speech unit to perform voice generation considering auditory characteristics.

The collection unit can further measure the user's skin conductance and analyze the data in the analysis unit. For example, when the user is tense, it detects an increase in skin conductance and analyzes the data in the analysis unit. Additionally, the collection unit can detect a decrease in skin conductance when the user is relaxed and analyze the data in the analysis unit. Furthermore, the collection unit can detect changes in skin conductance in real-time when the user is excited and analyze the data immediately in the analysis unit. This allows the collection unit to measure skin conductance for more accurate understanding of the user's emotional state.

Below is a brief explanation of the process flow in Example 2 of the Embodiment.

Step 1: The collection unit collects vital information. Vital information includes heart rate, brain waves, and body temperature. The collection unit collects vital information using brain wave sensors or heartbeat sensors. EEG sensors or fNIRS sensors are used as brain wave sensors, and photoplethysmographic sensors or electrical heartbeat sensors are used as heartbeat sensors.

Step 2: The analysis unit uses generative AI to analyze the vital information collected by the collection unit and understand emotions and thoughts. Deep learning models or natural language processing models are used as generative AI.

Step 3: The generation unit uses generative AI to generate words or sentences based on the analysis results obtained by the analysis unit. Text generation AI (e.g., LLM) or multimodal generation AI are used as generative AI.

Step 4: The speech unit uses voice generative AI to articulate the words or sentences generated by the generation unit. Voice synthesis models or text-to-speech conversion technologies are used as voice generative AI.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

### Second Embodiment

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices. Each of the multiple elements including the aforementioned collection unit, analysis unit, generation unit, and speech unit is realized by at least one of, for example, a smart device 14 and a data processing device 12. For example, the collection unit collects vital information using brain wave sensors or heartbeat sensors of the smart device 14. The analysis unit is realized by, for example, a specific processing unit 290 of the data processing device 12, which analyzes the collected vital information to understand emotions and thoughts. The generation unit is realized by, for example, a specific processing unit 290 of the data processing device 12, which generates words or sentences based on the analysis results. The speech unit articulates the generated words or sentences using, for example, the voice generative AI of the smart device 14. The correspondence between each unit and the device or control unit is not limited to the examples described above, and various modifications are possible.

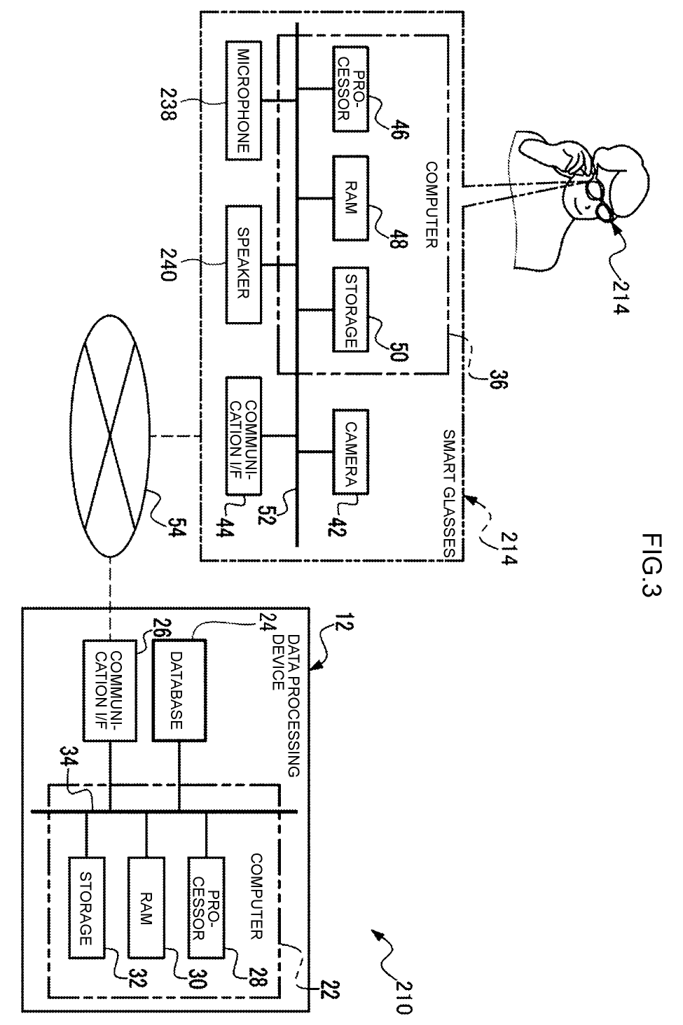

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal- Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

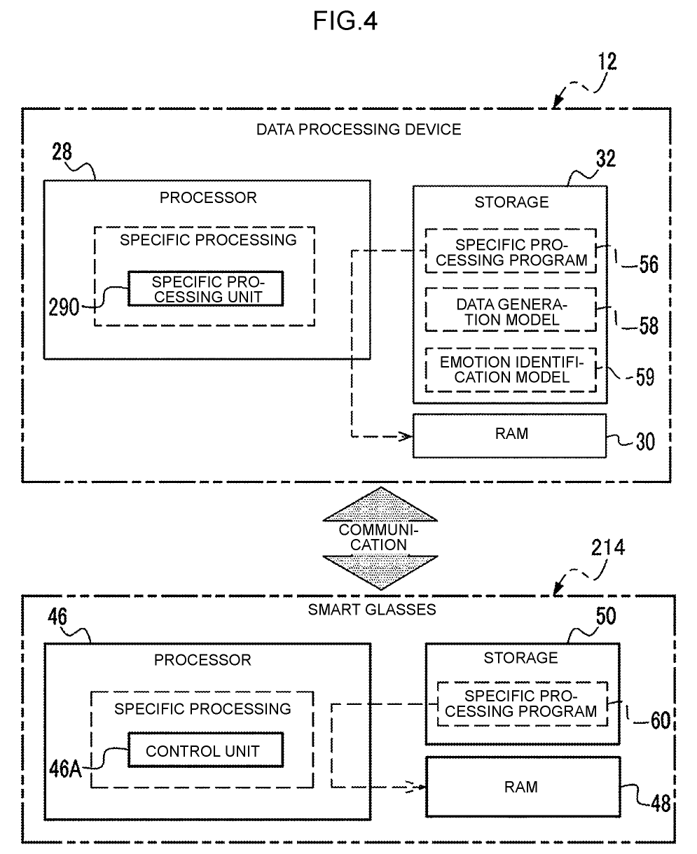

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59 and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVIV), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices. Each of the multiple elements including the aforementioned collection unit, analysis unit, generation unit, and speech unit is realized by at least one of, for example, smart glasses 214 and a data processing device 12. For example, the collection unit collects vital information using brain wave sensors or heartbeat sensors of the smart glasses 214. The analysis unit is realized by, for example, a specific processing unit 290 of the data processing device 12, which analyzes the collected vital information to understand emotions and thoughts. The generation unit is realized by, for example, a specific processing unit 290 of the data processing device 12, which generates words or sentences based on the analysis results. The speech unit articulates the generated words or sentences using, for example, the voice generative AI of the smart glasses 214. The correspondence between each unit and the device or control unit is not limited to the examples described above, and various modifications are possible.

### Third Embodiment

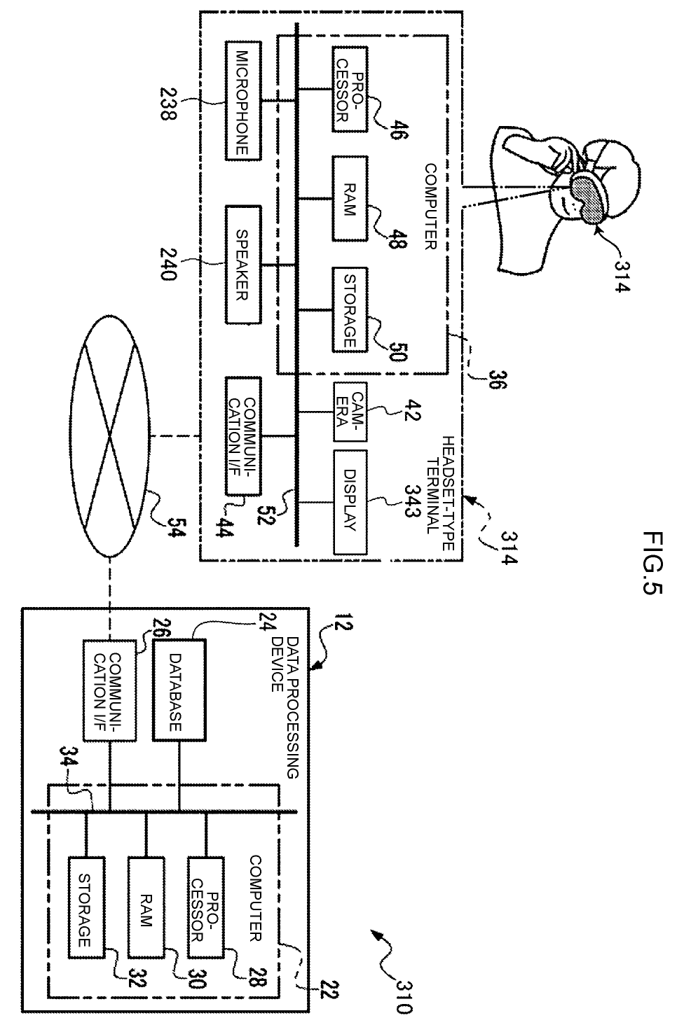

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal- Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

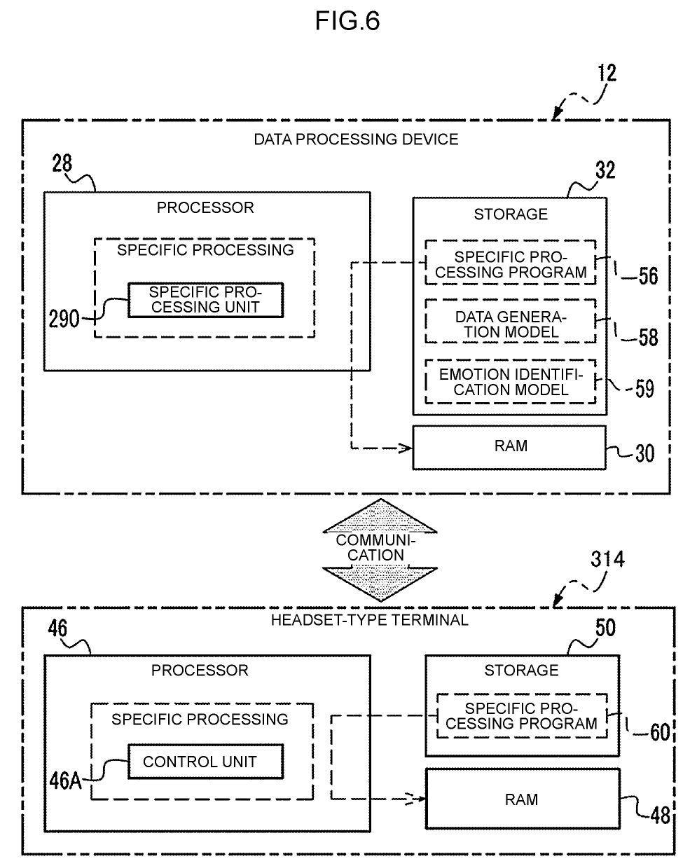

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59 and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVIV), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the multiple elements including the aforementioned collection unit, analysis unit, generation unit, and speech unit is realized by at least one of, for example, a headset-type terminal 314 and a data processing device 12. For example, the collection unit collects vital information using brain wave sensors or heartbeat sensors of the headset-type terminal 314. The analysis unit is realized by, for example, a specific processing unit 290 of the data processing device 12, which analyzes the collected vital information to understand emotions and thoughts. The generation unit is realized by, for example, a specific processing unit 290 of the data processing device 12, which generates words or sentences based on the analysis results. The speech unit articulates the generated words or sentences using, for example, the voice generative AI of the headset-type terminal 314. The correspondence between each unit and the device or control unit is not limited to the examples described above, and various modifications are possible.

### Fourth Embodiment

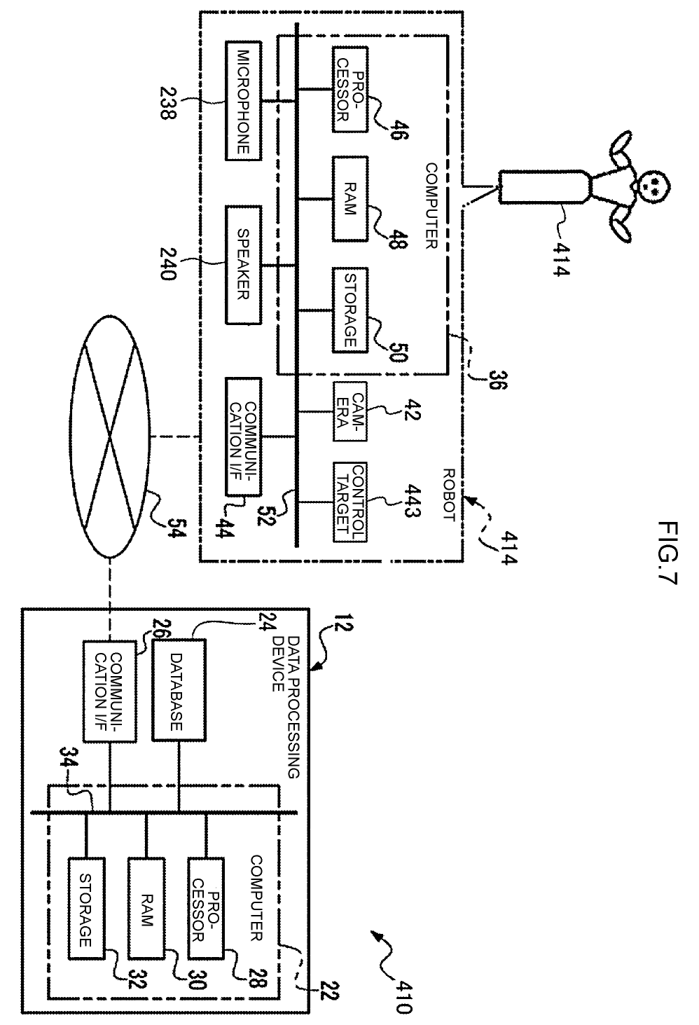

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

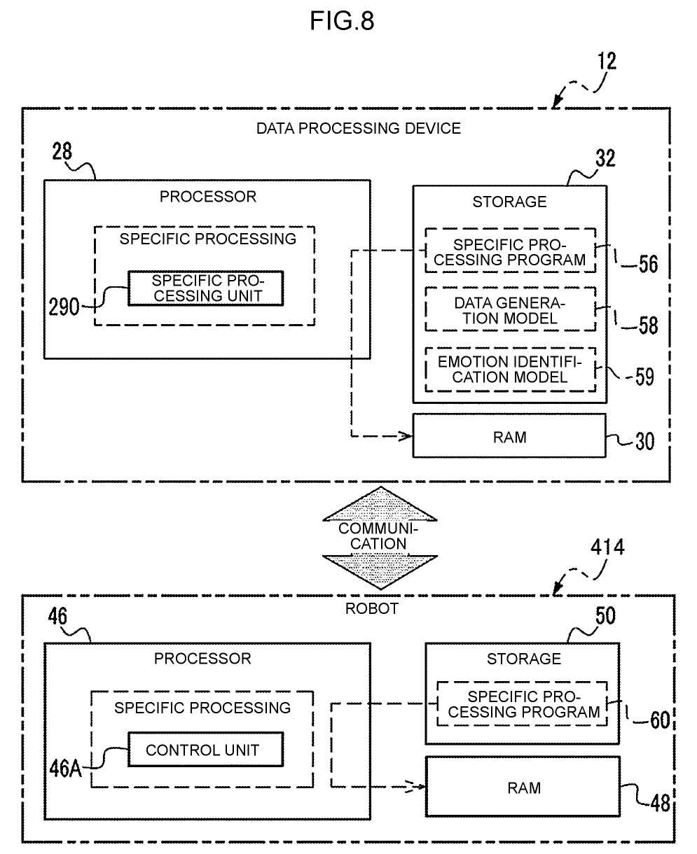

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59 and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVIV), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the multiple elements including the aforementioned collection unit, analysis unit, generation unit, and speech unit is realized by at least one of, for example, a robot 414 and a data processing device 12. For example, the collection unit collects vital information using brain wave sensors or heartbeat sensors of the robot 414. The analysis unit is realized by, for example, a specific processing unit 290 of the data processing device 12, which analyzes the collected vital information to understand emotions and thoughts. The generation unit is realized by, for example, a specific processing unit 290 of the data processing device 12, which generates words or sentences based on the analysis results. The speech unit articulates the generated words or sentences using, for example, the voice generative AI of the robot 414. The correspondence between each unit and the device or control unit is not limited to the examples described above, and various modifications are possible.

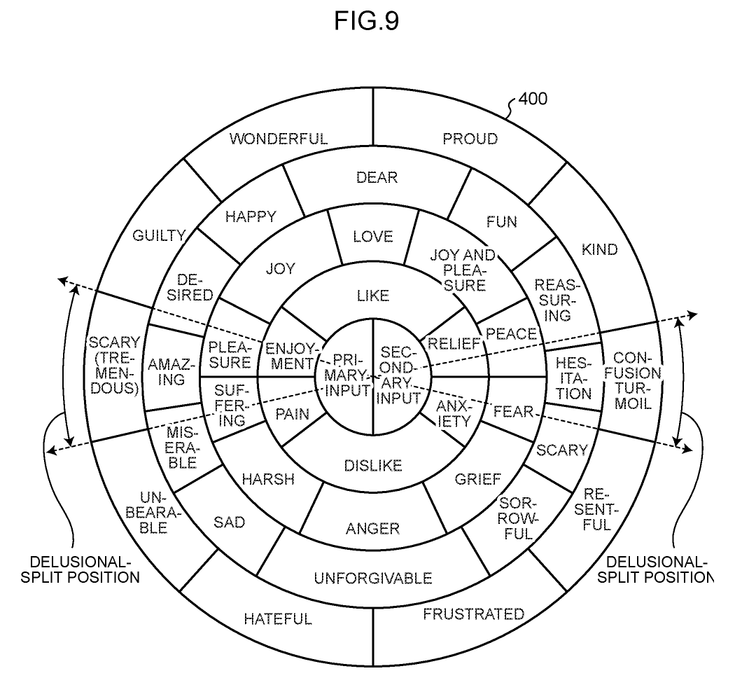

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, "pleasant" emotions are arranged, and on the lower side, "unpleasant" emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called "reactions," where sensations take precedence, are aligned. Additionally, in the right half of the emotion map, emotions belonging to the domain called "situations," where situational recognition takes precedence, are aligned.

In the emotion map, two emotions that promote learning are defined. One is a negative emotion around "repentance" or "reflection" on the situation side. In other words, when a negative emotion arises in the robot, like "I never want to feel this way again" or "I don't want to be scolded again." The other is an emotion around "desire" on the reaction side, which is positive. In other words, it is a positive feeling like "I want more" or "I want to know more."

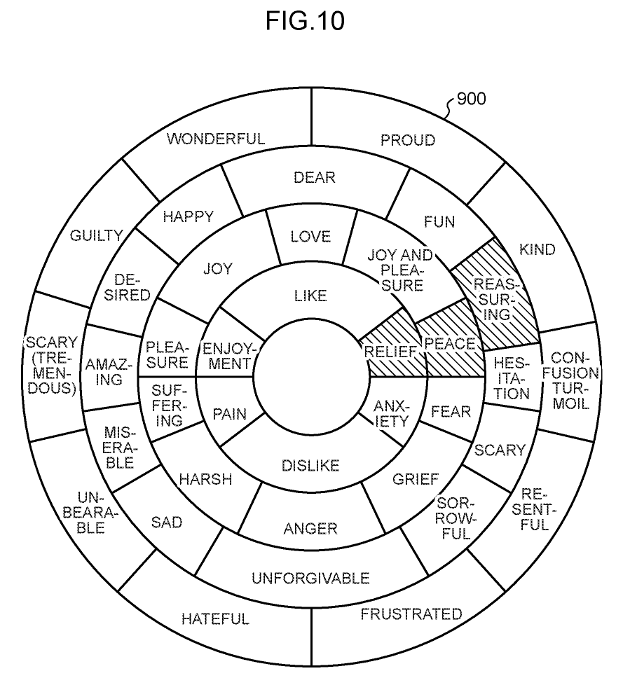

The emotion identification model 59 inputs user input into a pre-learned neural network, acquires emotion values indicating each emotion shown in the emotion map 400, and determines the user's emotions. This neural network is pre- learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like "reassured," "calm," and "confident" have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used. Additionally, the examples described above were explained by dividing into form example 1 and form example 2, but these may be combined.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Additional Note 1

A system including:

a collection unit that collects vital information;

an analysis unit that analyzes the information collected by the collection unit to understand emotions or thoughts;

a generation unit that generates a word or a sentence based on analysis result obtained by the analysis unit; and a speech unit that articulates the word or the sentence generated by the generation unit.

### Additional Note 2

The system according to Additional Note 1, wherein the collection unit includes a brain wave sensor or a heartbeat sensor.

### Additional Note 3

The system according to Additional Note 1, wherein the analysis unit analyzes emotions or thoughts by using a generative AI.

### Additional Note 4

The system according to Additional Note 1, wherein the generation unit generates a word or a sentence by using a generative AI.

### Additional Note 5

The system according to Additional Note 1, wherein the speech unit articulates a word or a sentence by using a voice generative AI.

### Additional Note 6

The system according to Additional Note 1, wherein the collection unit collects a brain wave by using a brain wave sensor.

### Additional Note 7

The system according to Additional Note 1, wherein the collection unit collects a heartbeat by using a heartbeat sensor.

### Additional Note 8

The system according to Additional Note 1, wherein the collection unit estimates user's emotions and adjusts a timing of vital information collection based on the estimated emotions.

### Additional Note 9

The system according to Additional Note 1, wherein the collection unit analyzes user's past vital information, and selects an appropriate collection method.

### Additional Note 10

The system according to Additional Note 1, wherein the collection unit filters based on a user's current health status or activity level during vital information collection.

### Additional Note 11

The system according to Additional Note 1, wherein the collection unit estimates user's emotions, and determines a priority of vital information to be collected based on the estimated emotions.

### Additional Note 12

The system according to Additional Note 1, wherein the collection unit prioritizes collection of relevant information by considering a user's geographical location during vital information collection.

### Additional Note 13

The system according to Additional Note 1, wherein the collection unit analyzes a user's social media activity and collects relevant information during vital information collection.

### Additional Note 14

The system according to Additional Note 1, wherein the analysis unit estimates user's emotions, and adjusts an expression method of analysis based on the estimated emotions.

### Additional Note 15

The system according to Additional Note 1, wherein the analysis unit adjusts a level of detail of analysis based on importance of vital information during analysis.

### Additional Note 16

The system according to Additional Note 1, wherein the analysis unit applies different analysis algorithms according to the category of vital information during analysis.

### Additional Note 17

The system according to Additional Note 1, wherein the analysis unit estimates user's emotions, and adjusts a length of analysis based on the estimated emotions.

### Additional Note 18

The system according to Additional Note 1, wherein the analysis unit determines a priority of analysis based on a collection timing of vital information during analysis.

### Additional Note 19

The system according to Additional Note 1, wherein the analysis unit adjusts an order of analysis based on relevance of vital information during analysis.

### Additional Note 20

The system according to Additional Note 1, wherein the generation unit estimates user's emotions, and adjusts an expression method of a word or a sentence to be generated based on the estimated emotions.

### Additional Note 21

The system according to Additional Note 1, wherein the generation unit adjusts a level of detail of generation based on an intensity of emotions during generation of a word or a sentence.

### Additional Note 22

The system according to Additional Note 1, wherein the generation unit applies different generation algorithms according to a type of emotions during generation of a word or a sentence.

### Additional Note 23

The system according to Additional Note 1, wherein the generation unit estimates user's emotions, and adjusts a length of a word or a sentence to be generated based on the estimated emotions.

### Additional Note 24

The system according to Additional Note 1, wherein the generation unit determines a priority of generation based on an occurrence timing of emotions during the generation of a word or a sentence.

### Additional Note 25

The system according to Additional Note 1, wherein the generation unit adjusts an order of generation based on relevance of emotions during generation of a word or a sentence.

### Additional Note 26

The system according to Additional Note 1, wherein the speech unit estimates user's emotions, and adjusts an expression method of speech based on the estimated emotions.

### Additional Note 27

The system according to Additional Note 1, wherein the speech unit adjusts a level of detail of speech based on importance of a word or a sentence generated during speech.

### Additional Note 28

The system according to Additional Note 1, wherein the speech unit applies different speech algorithms according to a category of a word or a sentence generated during speech.

### Additional Note 29

The system according to Additional Note 1, wherein the speech unit estimates user's emotions, and adjusts a length of speech based on the estimated emotions.

### Additional Note 30

The system according to Additional Note 1, wherein the speech unit determines a priority of speech based on an occurrence timing of a word or a sentence generated during speech.

### Additional Note 31

The system according to Additional Note 1, wherein the speech unit adjusts an order of speech based on relevance of a word or a sentence generated during speech.

1. A system comprising:
a collection unit that collects vital information;
an analysis unit that analyzes the information collected by the collection unit to understand emotions or thoughts;
a generation unit that generates a word or a sentence based on analysis result obtained by the analysis unit; and
a speech unit that articulates the word or the sentence generated by the generation unit.

2. The system according to claim 1, wherein the analysis unit analyzes emotions or thoughts using a generative AI.

3. The system according to claim 1, wherein the generation unit generates a word or a sentence by using a generative AI.

4. The system according to claim 1, wherein the speech unit articulates a word or a sentence by using a generative AI.

5. The system according to claim 1, wherein the collection unit collects a brain wave by using a brain wave sensor.

6. The system according to claim 1, wherein the collection unit collects a heartbeat by using a heartbeat sensor.

7. The system according to claim 1, wherein the collection unit estimates user's emotions, and adjusts a timing of vital information collection based on the estimated emotions.
